(19) United States
(12) Patent Application Publication
(10) Pub. No.: US 2009/0286869 A1
(43) Pub. Date: Nov. 19, 2009

(54) Flavanols and B-Type Procyanidins and Inflammation

(75) Inventor: Harold H. Schmitz, Bethesda, MD (US)

Correspondence Address: NADA JAIN, P.C., 560 White Plains Road, Suite 460, Tarrytown, NY, 10591, US

(73) Assignee: Mars, Inc., McLean, VA (US)

(21) Appl. No.: 12/084,408

(22) PCT Filed: Nov. 1, 2006
(86) PCT No.: PCT/US2006/042857

§ 371 Date: Apr. 30, 2008

Related U.S. Application Data
(60) Provisional application No. 60/732,280, filed on Nov. 1, 2005.

(51) Int. Cl. A61K 31/352

(52) U.S. Cl. 514/456

(57) ABSTRACT

The invention relates to compositions, and methods of use thereof, containing certain polyphenols such as flavanols, procyanidins and derivatives thereof for treating inflammation and/or inflammation-related or associated disease or condition, and/or for the relief of pain, in a subject sensitive to a selective cyclooxygenase-2 (COX-2) inhibitor and/or a subject sensitive to a COX-nonselective nonsteroidal anti-inflammatory drug (NSAID).

### FIELD OF THE INVENTION

The invention relates to compositions, and methods of use thereof, containing certain polyphenols such as flavanols, procyanidins and derivatives thereof for treating inflammation and/or inflammation-related or associated disease or condition, and/or methods for the relief of pain, in a subject sensitive to a selective cyclooxygenase-2 (COX-2) inhibitor and/or a subject sensitive to a COX-nonselective nonsteroidal anti-inflammatory drug (NSAID).

### BACKGROUND OF THE INVENTION

The procyanidins have attracted a great deal of attention in the fields of medicine and nutrition due to the wide range of their biological activities (e.g. U.S. Pat. Nos. 6,297,273; 6,670,390; 6,747,059; 6,524,630 and 6,638,971). Applicants have now discovered specific anti-inflammatory properties of procyanidins and derivatives thereof and their effect on cyclooxygenase-2 (COX-2) gene transcription, a key regulating enzyme in the biosynthesis of prostaglandins in humans and other mammals (Simmons et al., Pharmacol. Rev., 2004, 56:387437).

At least two distinct isoforms of cyclooxygenase are known: COX-1 and COX-2. COX-1 is constitutively expressed in many tissues, where it regulates physiological functions. In contrast, COX-2 is not normally expressed by most tissues, but is induced rapidly and transiently by proinflammatory mediators and mitogenic stimuli including cytokines, growth factors and tumor promoters. Up-regulation of COX-2 expression is observed at inflammatory sites, where it mediates the generation of prostaglandins responsible for pain and inflammation. The role of excessive inflammation as a critical factor in a wide range of human diseases is well established and therefore one of ordinary skill in the art will appreciate that the compounds of invention have utility in treating a diverse array of diseases, pathologies and conditions.

Most of the previously-known COX-2 inhibitors work primarily by blocking COX-2 enzyme activity either directly or indirectly. A disadvantage of inhibition at the enzyme level is that the loss of COX-2 enzyme activity is compensated for (by the body's natural response) by a bio-feedback loop which leads to an increased production of enzyme. The present inventive compounds offer a clear advantage as COX-2 synthesis is inhibited at the level of gene transcription thereby circumventing the formation of additional, undesirable COX-2 via the biofeedback loop mechanism. While the existing selective COX-2 inhibitors were found to be efficacious in blocking COX-2 activity and in reducing severe gastrointestinal events associated with use of nonselective NSAIDs, their safety following clinical administration has been questioned. The use of several commercially available selective COX-2 inhibitors has been shown to be associated with serious side effects, most notably those within the cardiovascular system such as myocardial infarction, strokes, and elevations in blood pressure.

### SUMMARY OF THE INVENTION

The invention relates to compositions, and methods of use thereof, containing certain polyphenols such as flavanols, procyanidins and derivatives thereof for treating inflammation and/or inflammation-related or associated disease or condition, and/or methods for the relief of pain, in a subject sensitive to a selective cyclooxygenase-2 (COX-2) inhibitor and/or a subject sensitive to a COX-nonselective nonsteroidal anti-inflammatory drug (NSAID).

In one aspect, the invention relates to a composition, such as a pharmaceutical, a food, a food additive, or a dietary supplement comprising the compound of the invention such as a flavanol, a procyanidin or a pharmaceutically acceptable salt or derivative thereof. The composition may optionally contain an additional COX-2 selective inhibitor and/or an additional COX-nonselective NSAID, or may be administered in combination with an additional COX-2 selective inhibitor and/or an additional COX-nonselective NSAID. Packaged products containing the above-mentioned composition and a label and/or instructions for use as described herein, e.g. to treat inflammation and/or inflammation-related or associated disease or condition, and/or for the relief of pain, in a subject sensitive to a selective COX-2 inhibitor and/or a subject sensitive to a COX-nonselective NSAID are also within the scope of the invention.

In another aspect, the invention relates to a method for treating inflammation and/or inflammation-related or associated disease or condition, and/or a method for the relief of pain, in a subject sensitive to a selective COX-2 inhibitor and/or a subject sensitive to a COX-nonselective NSAID, which comprises administering to a mammal, such as a human or a veterinary animal, an effective amount of a compound of the invention such as a flavanol, a procyanidin or a pharmaceutically acceptable salt or derivative thereof.

In a further aspect, the invention relates to a method comprising (i) profiling or diagnosing a subject for sensitivity to a selective COX-2 inhibitor and/or to a COX-nonselective NSAID, and (ii) treating the sensitive subject by administering an effective amount of the compound of the invention.

### BRIEF DESCRIPTION OF THE DRAWINGS

FIG. 1 shows the inhibitory effect of procyanidin dimer B2 on COX-2 mRNA transcription.

FIG. 2 shows the inhibitory effect of procyanidin dimer 132 on LPS-induced COX-2 protein expression.

FIGS. 3 A-B show that COX-2 enzyme activities are not inhibited by procyanidin dimer B2 (NS398 is a positive control).

FIGS. 4 A-C show the effects of B1 dimer, B2 dimer, (−)-catechin and (+)-epicatechin in comparison with A1 dimer (10 μM each) on the mRNA expression of COX-2 in LPS and fMLP-pretreated macrophages, and their potency comparison. n=3-4, Mean±SD. **P<0.01 versus control, ^{##}P<0.01 versus LPS, ^{$$}P<0.01 versus LPS+fMLP. The normal macrophages without LPS or fMLP treatment were used as controls.

### DETAILED DESCRIPTION

All patents, patent applications and references cited in this application are hereby incorporated herein by reference. In case of any inconsistency, the present disclosure governs.

The present invention relates to a compound, and a composition comprising an effective amount of the compound, having the following formula Aₙ, or a pharmaceutically acceptable salt or derivative thereof (including oxidation products, methylated derivatives and glucuronidated derivatives):

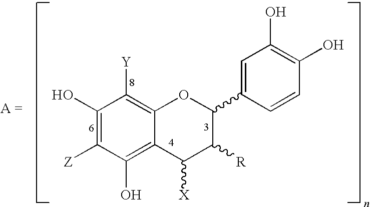

wherein

n is an integer from 2 to 18;

R and X each have either α or β stereochemistry;

R is OH, O-sugar or O-gallate;

the substituents of C-4, C-6 and C-8 are X, Z and Y, respectively, and bonding of monomeric units occurs at C-4, C-6 or C-8;

when any C-4, C-6 or C-8 is not bonded to another monomeric unit, X, Y and Z independently are hydrogen or a sugar; and

the sugar is optionally substituted with a phenolic moiety at any position, for instance, via an ester bond.

Monomeric units in the above formula may be bonded via 4→6α; 4→6β; 4→8α; and/or 4→8β linkages. The sugar is preferably a monosaccharide or a di-saccharide. The sugar may be selected from the group consisting of glucose, galactose, rhamnose, xylose, and arabinose. The phenolic moiety may be selected from the group consisting of caffeic, cinnamic, coumaric, ferulic, gallic, hydroxybenzoic and sinapic acids. Procyanidin derivatives may include esters such as the gallate esters (e.g. B2 dimer gallate); compounds derivatized with a saccharide moiety such as mono- or di-saccharide moiety (e.g. β-D-glucose), glucuronidated and methylated derivatives, and oxidation products. Oxidation products may be prepared as disclosed in U.S. Pat. No. 5,554,645, the relevant portions of which are incorporated herein by reference. Esters, for example esters with gallic acid, may be prepared using known esterification reactions, and for example as described in U.S. Pat. No. 6,420,572, the disclosure of which is hereby incorporated herein by reference. Methylated derivatives, such as 3′O-methyl-, 4′O-methyl-, and 3′O, 4′O-dimethyl-derivatives may be prepared, for example, as described in Cren-Olive et al., 2002, J. Chem. Soc. Perkin Trans. 1, 821-830, and Donovan et al., Journal of Chromatography B, 726 (1999) 277-283, the disclosures of which are hereby incorporated herein by reference. Glucuronidated products may be prepared as described in Yu et al, “A novel and effective procedure for the preparation of glucuronides,” Organic Letters, 2(16) (2000) 253-941, and as in Spencer et al, “”Contrasting influences of glucuronidation and O-methylation of epicatechin on hydrogen peroxide-induced cell death in neurons and fibroblasts,” Free Radical Biology and Medicine 31(9) (2001) 1139-46, both of which are hereby incorporated herein by reference. It should be noted that this disclosure applies to all formulas recited herein including the flavanols.

In certain embodiments, the invention relates to a compound, and the composition comprising an effective amount the compound having the formula Aₙ, or a pharmaceutically acceptable salt or derivative thereof (including oxidation products, methylated derivatives and glucuronidated derivatives):

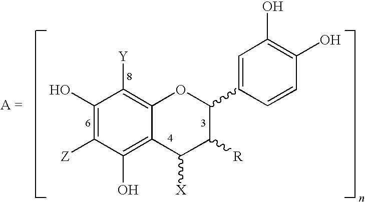

wherein

n is an integer from 2 to 18;

R and X each have either α or β stereochemistry;

R is OH;

the substituents of C-4, C-6 and C-8 are X, Z and Y, respectively, and bonding of monomeric units occurs at C-4, C-6 and C-8; and

when any C-4, C-6 or C-8 is not bonded to another monomeric unit, X, Y and Z are hydrogen.

Examples of the compounds useful for the products and in the methods of the invention include the compounds described herein wherein the integer n is 3 to 18; 2 to 12; 3 to 12; 2 to 5; 4 to 12; 5 to 12; 4 to 10; or 5 to 10. In some embodiments, the integer n is 2 to 4, for example 2 or 3. This disclosure applies to any compound of formula Aₙ herein.

In one embodiment, the invention relates to a compound, and a composition comprising an effective amount of the compound, having the following formula Aₙ, or a pharmaceutically acceptable salt or derivative thereof (including oxidation products, methylated derivatives and glucuronidated derivatives):

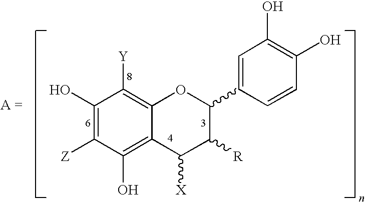

wherein

n is 2;

R and X each have either α or β stereochemistry;

R is OH, O-sugar or O-gallate;

the substituents of C-4, C-6 and C-8 are X, Z and Y, respectively, and bonding of monomeric units occurs at C-4, C-6 and C-8; and

when any C-4, C-6 or C-8 are not bonded to another monomeric unit, X, Y and Z independently are hydrogen or sugar; and

the sugar is optionally substituted with a phenolic moiety at any position, for instance, via an ester bond.

In another embodiment, the invention relates to a compound, and a composition comprising an effective amount of the compound, having the following formula Aₙ, or a pharmaceutically acceptable salt or derivative thereof (including oxidation products, methylated derivatives and glucuronidated derivatives):

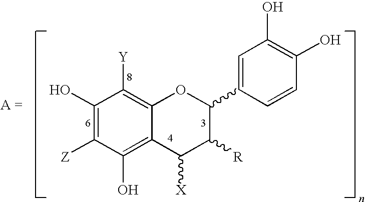

wherein

n is 2;

R and X each have either α or β stereochemistry;

R is OH;

the substituents of C-4, C-6 and C-8 are X, Z and Y, respectively, and bonding of monomeric units occurs at C-4, C-6 and C-8; and

when any C-4, C-6 or C-8 are not bonded to another monomeric unit, X, Y and Z are hydrogen.

Examples of dimers within the scope of the invention are dimers B1 [(−)-epicatechin-(4β-8)-(+)-catechin], B2 [(−)-epicatechin-(4β-8)-(−)-epicatechin] and B5 [(−)-epicatechin-(4β-6)-(−)-epicatechin].

B1 dimer [(−)-epicatechin-(4β-8)-(+)-catechin] has the following formula:

### B1 Dimer—epicatechin-(4-β-8)-catechin

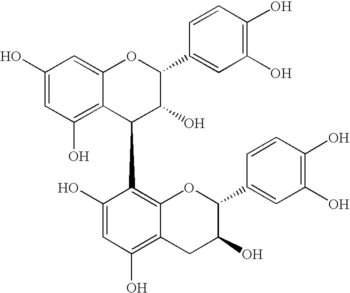

B2 dimer [(−)-epicatechin-(4β-8)-(−)-epicatechin] has the following formula:

### B2 Dimer—epicatechin-(4-β-8)-picatechin

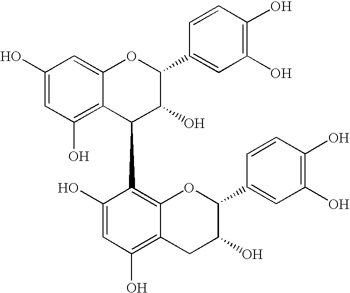

B5 dimer [(−)-epicatechin-(4β-6)-(−)-epicatechin] has the following formula:

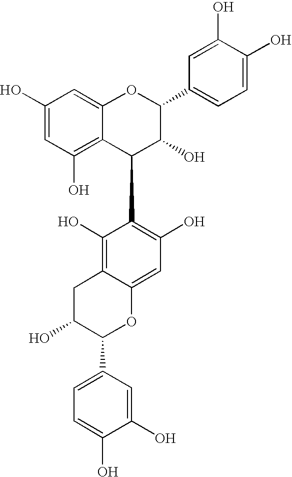

In other embodiments, the present invention relates to a flavanol or a flavan-3-ol. As used herein, the term “flavanol” or “flavan-3-ol” refers to a compound of the following formula:

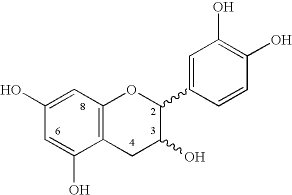

The invention also relates to a composition comprising an effective amount of the flavanol, or a pharmaceutically acceptable salt or derivative thereof (including oxidation products, methylated derivatives and glucuronidated derivatives). In some embodiments, the flavanol derivative is not a gallated derivative. Examples of and preparation of derivatives are as described above.

In certain embodiments, the flavanols of the above of formula may have beta stereochemistry at the C2 atom as shown below:

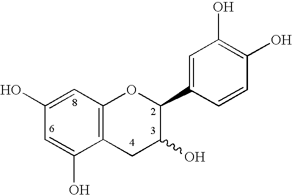

Examples of flavanols are (+)-epicatechin, (−)-epicatechin, (+)-catechin, and (−)-catechin.

### Methods of Use

The invention relates to methods of treating inflammation and/or inflammation-related or associated disease or condition, and/or to methods for the relief of pain, in a subject sensitive to a selective cyclooxygenase-2 (COX-2) inhibitor and/or a subject sensitive to a COX-nonselective nonsteroidal anti-inflammatory drug (NSAID). Any compound described in the application may be used to practice the methods described herein.

As used herein, “treatment” or “treating” means improving an existing medical condition, for example an inflammatory disease or condition, for example by slowing down the disease progression, prolonging survival, reducing the risk of death, and/or inducing a measurable reduction in inflammation.

As used herein, treatment of “inflammation and/or inflammation-related or associated disease or condition” refers to treatment of inflammation other than the inflammation associated with diseases or conditions of the vascular system (inclusive of the heart, the brain and the renal system). Examples of such diseases or conditions are: a gastrointestinal disease or condition other than GI complications of NSAIDs (e.g. inflammatory bowel diseases, Crohn's disease, regional enteritis, ulcerative colitis, diverticulitis, pancreatitis); a respiratory/pulmonary disease or condition (e.g. emphysema, acute respiratory distress syndrome, asthma, bronchitis, chronic obstructive pulmonary disease); a musculoskeletal disease or condition (e.g. arthritis including rheumatoid arthritis, osteoarthritis, gouty arthritis, juvenile arthritis, degenerative joint diseases, and spondyloarthropathies, muscle or joint strains or sprains, osteoporosis, loosening of artificial joint implants, myositis, polymyositis, bursitis, synovitis, ankylosing spondylitis, tendonitis); a dermal disease or condition (e.g. psoriasis, eczema, scleroderma, dermatitis, epidermolysis bullosa); an allergic disease or condition (e.g. allergic reactions, allergic contact hypersensitivity); pain associated with dysmenorrhea, menstrual cramps, headache (including migraine), toothache, low back and neck pain; ocular diseases or conditions (e.g. macular degeneration, conjunctivitis, corneal scarring, scleritis, ocular angiogenesis); a diabetes-associated condition (e.g. diabetic retinopathy, diabetic neuropathy, diabetic nephropathy, inflammatory conditions associated with type I and type II diabetes); fever (e.g. fever associated with influenza and other viral infections, rheumatic fever, common cold); systemic lupus erythematosis; inflammation-associated with burns; an inflammatory disease or condition affecting multiple organs (e.g. Sarcoidosis, Behcet's syndrome).

As used herein, a “subject sensitive to selective COX-2 inhibitor” is a subject in whom an existing health condition is exacerbated by, or an adverse health condition/reaction results from, the use of selective COX-2 inhibitor, or a subject for whom use of selective COX-2 inhibitor is contraindicated due to existence of previous health history of known risk factors which may increase the likelihood of adverse side effects associated with use of selective COX-2 inhibitors. Examples of these subjects are: elderly (e.g. age >65 for example >75); subjects with history of ischemic heart disease, hypertension or heart failure, subjects showing symptoms of an unstable coronary heart disease, subjects who have recently undergone heart surgery, subjects showing symptoms of an imminent cerebral ischemia, subjects with decompensated heart failure, subjects with uncontrolled arterial hypertension, subjects with previous history of NSAID-induced urticaria or angioedema, subjects with history of sulfonamide hypersensitivity (note: in these subjects only use of selective COX-2 inhibitors which have a sulfonamide group is contraindicated), subjects with previous history of NSAID-related nephrotoxicity, salt-depleted healthy subjects, elderly subjects (e.g. age >65 for example >75) with compromised renal function, pregnant women in third trimester of pregnancy, subjects with liver problems/hepatic dysfunction.

As used herein, a “subject sensitive to COX-nonselective NSAIDS” is a subject in whom an existing health condition is exacerbated or an adverse health condition/reaction results from the use of a COX-nonselective NSAID, or for whom use of COX-nonselective NSAID is contraindicated due to existence of previous health history of known risk factors which may increase the likelihood of adverse side effects associated with use of a COX non-selective NSAID. Examples of these subjects are: subjects with history of gastrointestinal complications (e.g. gastroduodenal perforations, ulcers and bleeding), elderly subjects (e.g. age >65 for example >75), subjects with history of NSAID-sensitive asthma or respiratory disease, subjects with history of NSAID-induced cutaneous reactions (e.g. urticaria, angeoedema, non-urticarial rash), subjects/children with chicken pox or influenza, subjects with NSAID-induced nephrotoxicity, subjects with liver failure, subjects with sulfonamide hypersensitivity (note: only use of NSAIDS with sulfonamide group is contraindicated), subjects undergoing treatment with anticoagulants, subjects undergoing treatment with corticosteroids, subjects with concurrent illnesses (e.g. rheumatoid arthritis, heart disease).

In certain embodiments, the invention provides a method of treating inflammation and/or inflammation-related or associated disease or condition, and/or a method for the relief of pain, which comprises administering to a human or a veterinary animal in need thereof an effective amount of a compound having the following formula Aₙ, or a pharmaceutically acceptable salt or derivative thereof (including oxidation products, methylated derivatives and glucuronidated derivatives):

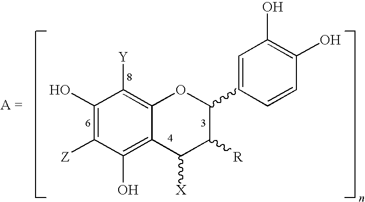

wherein

n is an integer from 2 to 18;

R and X each have either α or β stereochemistry;

R is OH, O-sugar or O-gallate;

the substituents of C-4, C-6 and C-8 are X, Z and Y, respectively, and bonding of monomeric units occurs at C-4, C-6 or C-8;

when any C-4, C-6 or C-8 is not bonded to another monomeric unit, X, Y and Z independently are hydrogen or a sugar; and

the sugar is optionally substituted with a phenolic moiety at any position, for instance, via an ester bond; and

wherein the subject is sensitive to a selective COX-2 inhibitor and/or a COX-nonselective NSAID.

For example, the above method may involve use of a compound Aₙ, or a pharmaceutically acceptable salt or derivative thereof (including oxidation products, methylated derivatives and glucuronidated derivatives), wherein R is OH, and when any C-4, C-6 or C-8 is not bonded to another monomeric unit, X, Y and Z are hydrogen. Examples of suitable sugars are as described above. Examples of phenolic moieties are as described above. Examples of derivatives are as described above.

The invention also encompasses a method of treating inflammation and/or inflammation-related or associated disease or condition, and/or a method for the relief of pain, which comprises administering to a human or a veterinary animal an effective amount of a compound having the formula Aₙ, or a pharmaceutically acceptable salt or derivative thereof (including oxidation products, methylated derivatives and glucuronidated derivatives):

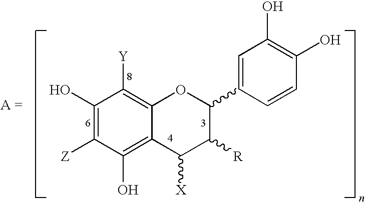

wherein

n is 2;

R and X each have either α or β stereochemistry;

R is OH, O-sugar or O-gallate;

the substituents of C-4, C-6 and C-8 are X, Z and Y, respectively, and bonding of monomeric units occurs at C-4, C-6 or C-8;

when any C-4, C-6 or C-8 is not bonded to another monomeric unit, X, Y and Z independently are hydrogen or a sugar; and

the sugar is optionally substituted with a phenolic moiety at any position, for instance, via an ester bond; and

wherein the subject is sensitive to a selective COX-2 inhibitor and/or a COX-nonselective NSAID.

In one of the embodiments, the invention encompasses a method of treating inflammation and/or inflammation-related or associated disease or condition, and/or a method for the relief of pain, which comprises administering to a human or a veterinary animal in need thereof an effective amount of a compound having the formula Aₙ, or a pharmaceutically acceptable salt or derivative thereof (including oxidation products, methylated derivatives and glucuronidated derivatives):

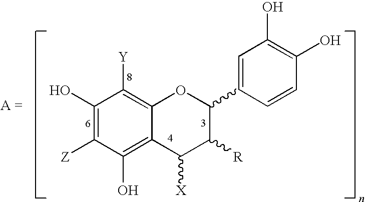

wherein

n is 2

R and X each have either α or β stereochemistry;

R is OH;

the substituents of C-4, C-6 and C-8 are X, Z and Y, respectively, and bonding of monomeric units occurs at C-4, C-6 and C-8; and

when any C-4, C-6 or C-8 are not bonded to another monomeric unit, X, Y and Z are hydrogen; and

wherein the subject is sensitive to a selective COX-2 inhibitor and/or a COX-nonselective NSAID.

In certain embodiments, the invention provides a method of treating inflammation and/or inflammation-related or associated disease or condition, and/or a method for the relief of pain, which comprises administering to a human or a veterinary animal in need thereof an effective amount of a flavanol or a flavan-3-ol of the following formula:

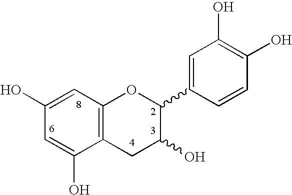

or a pharmaceutically acceptable salt or derivative thereof (including oxidation products, methylated derivatives and glucuronidated derivatives) wherein the subject is sensitive to a selective COX-2 inhibitor and/or a COX-nonselective NSAID. Examples of derivatives are as described above, and in some embodiments, the flavanol derivative is not a gallated derivative.

In certain embodiments, the flavanol of the above of formula may have the following structure:

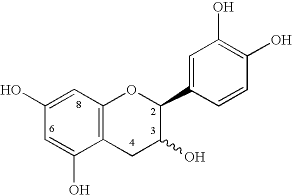

Examples of the compounds for use in the methods described herein are epicatechin, (−)-epicatechin, (+)-catechin, and (−)-catechin, and dimers B1, B2, and B5.

The present compounds may be administered alone, or as a combination therapy with other COX-2 selective inhibitor(s), most of which primarily target the COX-2 enzyme activity. Examples of COX-2 inhibitors include: meloxicam, etodolac, nimesulide, flosulide, lumiracoxib, celecoxib, valdecoxib, rofecoxib, deracoxib, parecoxib, etoricoxib, darbufelone, and meclofenamate esters and amides.

The present compounds may also be administered in combination with COX-nonselective NSAID(s). Examples of COX-nonselective NSAIDs include: nabumetone, meclofenamic acid, mefenamic acid, ibuprofen, flurbiprofen, suprofen, ketoprofen, naproxen, piroxicam, tenoxicam, phenylbutazone, diclofenac, ketorolac, tolmetin, indomethacin, sulindac, and acetamiophen.

When used for the above-mentioned combination therapies, COX-2 selective inhibitor and/or COX-nonselective NSAID may be administered in reduced amounts, i.e., amounts lower than when they are administered alone thereby reducing the side effects of these compounds.

The present compounds may be administered, in some embodiments, in combination with and/or to enhance responsiveness of immunomodulatory agents (other than NSAIDs and selective COX-2 inhibitors). Examples of such agents include: biological therapeutic agents (e.g. tumor necrosis factor (TNF)-α inhibitors such as anti-TNF monoclonal antibodies and TNF receptors (examples of TNF-blockers include entanercept, infliximab and adalimumas), matrix metalloproteinase inhibitors, aggrecanase inhibitors, tumor necrosis alpha converting enzyme (TACE) inhibitors, leukotriene receptor antagonists, interleukin-1 (IL-1) processing and release inhibitors, prostaglandin inhibitors such as PGD-, PGF-, PGI₂ and PGE-receptor antagonists); anti-osteoporosis agents (e.g. raloxifene, lasofoxifene, droloxifene, zoledronate, alendronate, risedronate, ibandronate, etidornate, teriparatide, miacalcin); anti-gout agents (e.g. colchicines, xanthine oxidase inhibitors, uricosuric agents such as probenecid, sufinpyrazole and benbromane); anti-arthritis drugs (e.g. leflunomide, oral gold, sulfasalazine, mycophenolate, injectable gold, cyclosporine, cyclophosphamide, azathioprine, chlorambucil, methotrexate, minocycline, cuprimine, hydroxychloroquine); anti-inflammatory glucocorticoids (e.g. betamethasone, cortisone, dexamethasone, hydrocortisone, methylprednisolone, prednisolone, prednisone); antacids; histamine (H2) receptor blockers (e.g. Axid®, Pepcid®, Zantac®); proton pump inhibitors (e.g. rabeprazole, esomeprazole, lansoprazole, omeprazole, pantoprazole). For that purpose, dosage forms other than pharmaceuticals, e.g. dietary supplements and foods, may also be used (e.g. chondroprotective nutraceuticals such as polysulfated glycosaminoglycan, glucosamine, chondroitin sulfate, hyaluronic acid, and pentosan polysulfate).

The methods described herein may be used in a human or a veterinary animal, such as a dog, a cat, and a horse.

Thus, the following uses are within the scope of the invention. Use of a flavanol and/or a compound Aₙ, or a pharmaceutically acceptable salt or derivative thereof (including oxidation products, methylated derivatives and glucuronidated derivatives), as defined above, in the manufacture of a medicament, food, nutraceutical or dietary supplement for inhibiting COX-2 expression in a subject sensitive to a selective COX-2 inhibitor and/or a COX-nonselective NSAID. Use of a flavanol or a compound of formula Aₙ, or a pharmaceutically acceptable salt or derivative thereof (including oxidation products, methylated derivatives and glucuronidated derivatives), as defined herein, in the manufacture of a medicament, food, nutraceutical or dietary supplement for use in treating inflammation and/or inflammation-related or associated disease or condition in a subject sensitive to a selective COX-2 inhibitor and/or a COX-nonselective NSAID.

The above described methods may further comprise determining the effectiveness of the treatment, for example, by measuring the level of COX-2 expression in a tissue sample using techniques known in the art.

The advantage of the present invention is that it can offer a personalized medicine approach to the treatment of inflammation and/or inflammation-related or associated disease or condition. Each patient/subject can be profiled or diagnosed for his/her sensitivity to COX-2 selective inhibitors and/or COX-nonselective NSAIDs and treating according to the methods described herein. It will be understood by a person of skill in the art that the sensitive subjects can be identified as described herein and as is known in the art.

The effective amount may be determined by a person of skill in the art using the guidance provided herein and general knowledge in the art. For example, the effective amount may be such as to achieve a physiologically relevant concentration in the body of a mammal. Such a physiologically relevant concentration may be at least 20 nanomolar (nM), preferably at least about 100 nM, and more preferably at least about 500 nM. In one embodiment, at least about one micromole in the blood of the mammal, such as a human, is achieved. The compounds as defined herein, may be administered at from about 50 mg/day to about 1000 mg/day, preferably from about 100-150 mg/day to about 900 mg/day, and most preferably from about 300 mg/day to about 500 mg/day. However, amounts higher than stated above may be used. The amounts may be measured as described in Adamson, G. E. et al., J. Ag. Food Chem.; 1999; 47 (10) 4184-4188, hereby incorporated herein by reference.

The treatment/administration may be continued as a regimen, i.e., for an effective period of time, e.g., daily, monthly, bimonthly, biannually, annually, or in some other regimen, as determined by the skilled medical practitioner for such time as is necessary. The administration may be continued for at least a period of time required to reduce inflammation to therapeutically relevant levels. Preferably, the composition is administered daily, most preferably two or three times a day, for example, morning and evening to maintain the levels of the effective compounds in the body of the mammal. To obtain the most beneficial results, the composition may be administered for at least about 30, or at least about 60 days. These regimens may be repeated periodically.

Compositions and Formulations

The compounds of the invention may be administered as a pharmaceutical, a food, a food additive, or a dietary supplement.

As used herein, a “pharmaceutical” is a medicinal drug. See Merriam-Webster's Collegiate Dictionary, 10th Edition, 1993. A pharmaceutical may also be referred to as a medicament. A “food” is a material containing protein, carbohydrate and/or fat, which is used in the body of an organism to sustain growth, repair and vital processes and to furnish energy. Foods may also contain supplementary substances, for example, minerals, vitamins and condiments. See Merriam-Webster's Collegiate Dictionary, 10th Edition, 1993. The term food includes a beverage adapted for human or animal consumption. As used herein a “food additive” is as defined by the FDA in 21 C.F.R 170.3(e)(1) and includes direct and indirect additives. As used herein, a “dietary supplement” is a product (other than tobacco) that is intended to supplement the diet that bears or contains the one or more of the following dietary ingredients: a vitamin, a mineral, an herb or other botanical, an amino acid, a dietary substance for use by man to supplement the diet by increasing the total daily intake, or a concentrate, metabolite, constituent, extract or combination of these ingredients. The above compositions may be prepared as is known in the art.

The compositions may contain a carrier, a diluent, or an excipient. Depending on the intended use, the carrier, diluent, or excipient may be chosen to be suitable for human or veterinary use, food, additive, dietary supplement or pharmaceutical use. The composition may optionally contain an additional anti-inflammatory agent. Also depending on use, a person of skill in the art may select the degree of purity of the compound of the invention. For example, when used to prepare pharmaceutical dosage forms, the compound should be as pure as commercially possible, while when preparing food, additive, or supplement, less pure or mixtures of compounds (e.g. plant extracts) may be used.

The compound of the invention may be “isolated and purified,” i.e., it may be separated from compounds with which it naturally occurs (e.g. when the compound is of natural origin), or it may be synthetically prepared, in either case such that the level of contaminating compounds and/or impurities does not significantly contribute to, or detract from, the effectiveness of the compound. For example, an “isolated and purified B2 dimer” is separated from B5 dimer, with which it may occur in nature (e.g. in cocoa bean), to the extent achievable by the available commercially viable purification and separation techniques. Such compounds are particularly suitable for pharmaceutical applications.

The compound may also be less pure, i.e., “substantially pure,” i.e., it may possess the highest degree of homogeneity achievable by available purification, separation and/or synthesis technology but need not be separated from the like compounds. As used herein, “the like compounds” are the compounds having the same degree of polymerization. For example, a “substantially pure dimer” refers to a mixture of dimers (e.g. B2 and B5, as it would occur in a cocoa extract fraction). While less suitable for pharmaceutical applications, such “substantially pure” compounds may be utilized for food, food additive and dietary supplement applications.

In some embodiments, the compound of the invention is at least 80% pure, at least 85% pure, at least 90% pure, at least 95% pure, at least 98% pure, or at least 99% pure. Such compounds are particularly suitable for pharmaceutical applications.

Pharmaceuticals containing the inventive compounds, optionally in combination with another anti-inflammatory agent, may be administered in a variety of ways such as orally, sublingually, bucally, nasally, rectally, intravenously, parenterally and topically. A person of skill in the art will be able to determine a suitable mode of administration to maximize the delivery of the compound of formula Aₙ, and optionally another anti-inflammatory treating agent, to the site of the inflammation. Thus, dosage forms adapted for each type of administration are within the scope of the invention and include solid, liquid and semi-solid dosage forms, such as tablets, capsules, gelatin capsules (gelcaps), bulk or unit dose powders or granules, emulsions, suspensions, pastes, creams, gels, foams or jellies. Sustained-release dosage forms are also within the scope of the invention. Suitable pharmaceutically acceptable carriers, diluents, or excipients are generally known in the art and can be determined readily by a person skilled in the art. The tablet, for example, may comprise an effective amount of the compound of the invention and optionally a carrier, such as sorbitol, lactose, cellulose, or dicalcium phosphate.

The foods comprising the compounds described herein and optionally another anti-inflammatory agent may be adapted for human or veterinary use, and include pet foods. The food may be other than a confectionery, for example, a beverage (e.g. cocoa flavored beverage). A confectionery such as a standard of identity (SOI) and non-SOI chocolate, such as milk, sweet and semi-sweet chocolate including dark chocolate, low fat chocolate and a candy which may be a chocolate covered candy are also within the scope of the invention. Other examples include a baked product (e.g. brownie, baked snack, cookie, biscuit) a condiment, a granola bar, a toffee chew, a meal replacement bar, a spread, a syrup, a powder beverage mix, a cocoa or a chocolate flavored beverage, a pudding, a rice cake, a rice mix, a savory sauce and the like. If desired, the foods may be chocolate or cocoa flavored. Food products may be chocolates and candy bars, such as granola bars, containing nuts, for example, peanuts, walnuts, almonds, and hazelnuts.

The compounds for use in the present invention may be of natural origin, for example, derived from a cocoa bean or another natural source known to a person of skill in the art, or prepared synthetically. A person of skill in the art may select natural or synthetic polyphenol based on the use and/or availability or cost.

The compounds may be included in the composition in the form of a cocoa ingredient, for example, chocolate liquor included in chocolate, or may be added independently of cocoa ingredients, for example, as an extract, extract fraction, isolated and purified individual compound, pooled extract fractions or a synthetically prepared compound. The term “cocoa ingredient” refers to a cocoa solids-containing material derived from shell-free cocoa nibs such as chocolate liquor and partially or fully-defatted cocoa solids (e.g. cake or powder). The extraction and purification may be conducted as described in U.S. Pat. Nos. 5,554,645 and 6,670,390 to Romanczyk et al., which is hereby incorporated herein by reference.

Synthetic procyanidins may also be used and are prepared by methods known in the art and as described, for example in, U.S. Pat. Nos. 6,420,572; 6,156,912; and 6,864,377, the relevant portions of each of which are hereby incorporated herein by reference.

A flavanol having beta stereochemistry at the C-2 atom may be prepared by thermally treating (in an aqueous solution) a flavanol having alpha stereochemistry at C-2 atom (which are commonly found in nature, for example in cocoa) to cause rotation about the C2 atom resulting in beta stereochemistry at the C-2 atom.

Preparation of a flavanol having beta stereochemistry at the C-2 atom may be conducted according to the following scheme 1 (and as described in Freudenberg, K. and Purrmann, L. (1924). Raumisomere Catechin IV. Liebig's Annalen, 437, 472-85; Fredenberg, K., Bohme, L. and Purrmann, L. (1922). Raumisomere Catechin II. Ber. Dscht. Chem. Ges., 55, 1734-47, the disclosures of which are hereby incorporated herein by reference):

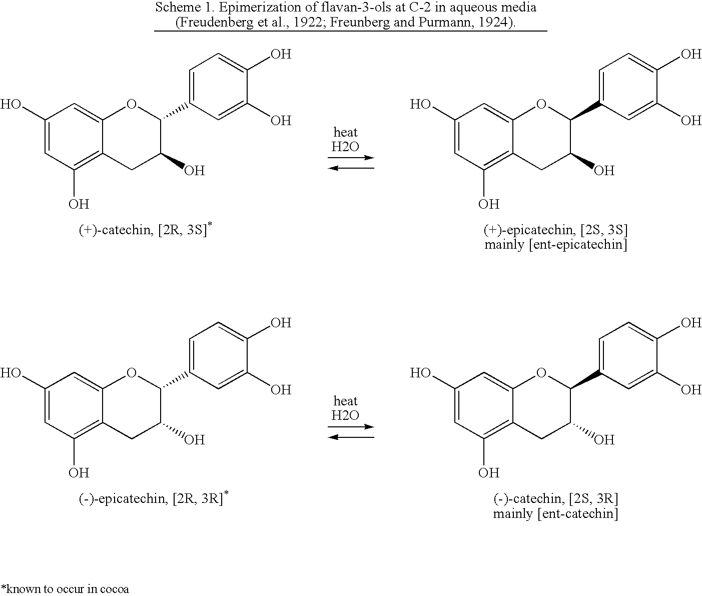

The lower the temperature, the longer the exposure required to cause rotation about the C-2 atom. For examples, such temperatures and times may be at least 40° C., more preferably at least 50° C., for at least 10 hours, more preferably at least 24 hours, or more preferably at least 48 hours; or at least 60° C., at least 70° C., at least 80° C., at least 90° C., at least 100° C., at least 110° C., or at least 120° C., each for at least five, or at least 10, 15 or 20 minutes. For example, the compound may be treated at 120° C. for 10 minutes, or 120° C. for 20 minutes. Other temperature/time combinations are also effective and the skilled artisan may determine such without undue experimentation using general knowledge in the art and the guidance provided herein. Known techniques, such as HPLC/MS analysis may be used to monitor the success of the reaction.

A daily effective amount of the compound of the invention may be provided in a single serving in case of a food or a single dosage in case of a pharmaceutical or a dietary supplement. For example, a confectionery (e.g. chocolate) may contain at least about 100 mg/serving (e.g. 150-200,200400 mg/serving).

The dietary supplement containing cocoa flavanol and/or procyanidin, and optionally another anti-inflammatory treating agent, may be prepared using methods known in the art and may comprise, for example, nutrients such as dicalcium phosphate, magnesium stearate, calcium nitrate, vitamins, and minerals.

Further within the scope of the invention is an article of manufacture such as a packaged product comprising the composition of the invention (e.g. a food, a dietary supplement, a pharmaceutical) and a label indicating the presence of, or an enhanced content of, the inventive compounds or directing use of the composition to treat inflammation and/or inflammation-related or associated disease or condition, and/or for the relief of pain, in a subject sensitive to a selective COX-2 inhibitor and/or a subject sensitive to a COX-nonselective NSAID. The packaged product may contain the composition and the instructions for use to treat inflammation and/or inflammation-related or associated disease or condition, and/or for the relief of pain, in a subject sensitive to a selective COX-2 inhibitor and/or a subject sensitive to a COX-nonselective NSAID. The label and/or instructions for use may refer to any of the methods of use described in this application.

The invention also relates a method of manufacturing an article of manufacture comprising any of the compositions described herein, packaging the composition to obtain an article of manufacture and instructing, directing or promoting the use of the composition/article of manufacture for any of the uses described herein. Such instructing, directing or promoting includes advertising.

The invention is further described in the following non-limiting examples.

### EXAMPLES

### Example 1

### Effect of Procyanidin B2 on COX-2 Expression

### Materials

Procyanidin dimer B2 was prepared from cocoa by solvent extraction, using gel permeation chromatography, followed by further purification/isolation of a dimer enriched fraction using Normal-Phase HPLC (described in detail in Adamson et al., J. Ag. Food Chem., 1999, 47 (10):4184-4188), see also U.S. Pat. No. 5,554,645, both of which are hereby incorporated herein by reference. This material was then passed over a C18 column to further enrich B2 dimer (98.3%) in the fraction which was used in the experiments described below.

Phorbol 12-myristate 13-acetate (PMA), Lipopolysaccharides (LPS, from Escherichia coli serotype 0111: B4) and NS398 (a selective COX-2 inhibitor) were purchased from Sigma (St. Louis, Mo.). RPMI 1640, L-glutamine, HEPES, 2-mercaptoethanol, fetal bovine serum, and penicillin/streptomycin were purchased from Gibco BRL (Grand Island, N.Y.). Anti-COX-2 was purchased from Santa Cruz Biotechnology Inc (Santa Cruz, Calif.). Anti-ERK, anti-JNK, anti-p38 MAPK, and their phosphor antibodies were purchased from Cell signaling technology (Beverly, Mass.). Alexa Fluor 488 goat anti-mouse IgG was purchased from Molecular Probes (Eugene, Oreg.). SuperSignal West Pico chemiluminescent substrate and PGE₂-specific RIA kit was purchased from Beijing East Asia Institute of Immunology (Beijing CN). All other chemicals used were in the purest form available commercially.

### Cell Culture

Human monocytic THP-1 cells from acute monocytic leukemia (American Type Culture Collection, Manassas, Va.) in RPMI 1640 medium (Life Technologies, Rockville, Md.), with 4.5 g/L glucose, 10 nM HEPES, 1 mM sodium pyruvate, and 50 μM 2-ME supplemented with 10% FBS, were cultured under a humidified 5% CO₂ atmosphere at 37° C. For differentiation, THP-1 cells were plated at 1×10⁶ cells/ml in the medium containing 100 nM PMA and allowed to adhere for 48 h, after which they were fed with PMA-free medium and cultured for 24 h prior to use. LPS was used at concentration of 1 μg/ml in the medium. sf9 cells were cultured in monolayer at 28° C. in Grace's supplemented medium with 10% heat-inactivated fetal bovine serum.

### Determination of COX-2 Enzyme Activity

The effect of procyanidin dimer B2 and NS398, a selective COX-2 inhibitor (positive control), on the activity of COX-2 was measured using baculovirus-expressed recombinant human COX-2 enzyme as previously described (Zhang et al., Acta. Pharmacol. Sin. 25(8):1000-1006, 2004). Briefly, 24 h after infecting sf9 cells with hCOX-2 recombinant baculovirus, the cells were collected and washed in HHBS. The assays were performed as follows. One milliliter of Hank's solution containing 1×10⁵ COX-2 expressing cells plus 9×10⁵ uninfected sf9 cells was dispensed per well of 24-well polypropylene plates. B2, NS398, or DMSO vehicle (10 μL) was added to the appropriate well containing the cell suspension. Following a 15-min drug or DMSO preincubation at 37° C., the cells were challenged with 10 μmol/L arachidonic acid

(Sigma) in ethanol and incubated for 10 min. Reactions were terminated by the addition of 100 μL of 1 mol/L HCl, neutralized with 100 μL of 1 mol/L NaOH. The cells were pelleted for 10 min at 300×g and the levels of PGE₂ in the supernatant were determined by a PGE₂-specific RIA (Beijing East Asia Institute of Immunology). The concentration of PGE₂ was then determined by interpolation from a standard curve and inhibition calculated by comparison of the PGE₂ production by drug-treated cells (B2 and NS398) with that of DMSO-treated cells.

Real-Time Quantitative Reverse Transcriptase-Polymerase Chain Reaction (RT-PCR) to Assess COX-2 mRNA Expression

Total RNA was extracted from macropahges with TRIzol reagent (Invitrogen Corporation, Carlsbad, Calif.). Real-time quantitative RT-PCR was performed using the Opticon 2 (MJ Research Inc., Waltham, Mass.). Sequence specific PCR primers for COX-2 [accession no. NM_{—}000963; forward primer: 5′-GGGCAAAGACTGCGAAGAAG-3′ [SEQ ID NO: 1]; reverse primer: 5′-CCCATGTGACGAAATGACTG-3′ [SEQ ID NO: 2]] and GAPDH [accession no. NM_{—}002046; forward primer: 5′-ACGGATTTGGTCGTATTGGG-3′ [SEQ ID NO: 3]; reverse primer: 5′-CGCTCCTGGAAGATGGTGAT-3′ [SEQ ID NO: 4]] were designed using the Primer Premier software version 5.00. Standard curves were run on the same plate and the relative standard curve method was used to calculate the relative gene expression.

### Western Blot Analysis

5×10⁶ cells were resuspended in modified RIPA lysis buffer (Tris-HCl 50 mM, pH 7.4 NaCl 150 mM, EDTA 1 mM, Na-deoxycholate 0.25%, NP-40 1%, PMSF 1 mM, Na₃VO4 1 mM, NaF 1 mM, Aprotinin 10 μg/ml, leupeptin 5 μg/ml, pepstatin 5 μg/ml), and lysed cells on ice for 45 min. The lysate was centrifuged at 14,000×g for 15 min to sediment the particulate materials. The protein concentration of the supernatant was measured by the method of Lowry (Lowry et al., J. Biol. Chem. 193:265-267, 1951). Samples were electrophoresed in SDS/PAGE gels and separated proteins were transferred onto a PVDF membrane. The blots were blocked with 5% non-fat dry milk in Tris-buffered saline (TBS) for 1 h at room temperature and subsequently incubated overnight at 4° C. with primary antibodies diluted (1:1000) in TBST [TBS, 1% (v/v) Tween 20 and 5% (w/v) BSA]. Following three washes of 10 min each with TBST, the blots were incubated with horseradish peroxidase-conjugated secondary antibodies in blocking buffer for 1 h at room temperature. After three washes with TBST, the blots were developed with chemiluminescence reagent and exposed to X-ray film (Kodak XAR5, Eastman Kodak, Rochester, N.Y., U.S.A.).

### Results

### Procyanidin Dimer B2 Inhibits LPS-Induced Increases in the Transcription and Expression of COX-2 Protein

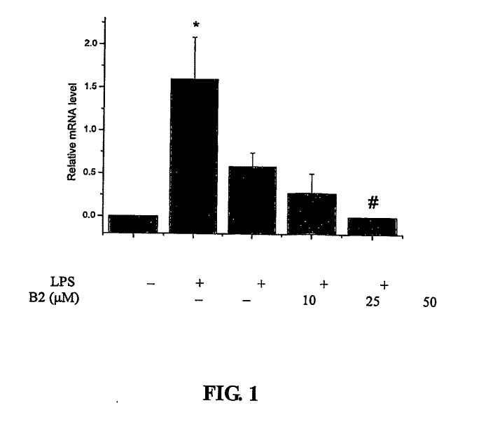

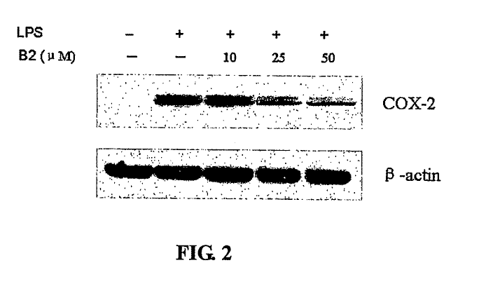

Treatment of differentiated THP-1 cells with 1 μg/ml LPS led to a dramatic increase in COX-2 transcription (FIG. 1). When THP-1 cells were exposed to dimer B2 and LPS for 4 h, the levels of mRNA for COX-2 were reduced in a concentration-dependent manner (FIG. 1). In a separate experiment (30 min cell pre-pretreatment with B2 followed by 4 hour LPS treatment), dimer B2, at a dose of 50 μM, inhibited COX-2 protein expression as illustrated by the Western blot in FIG. 2.

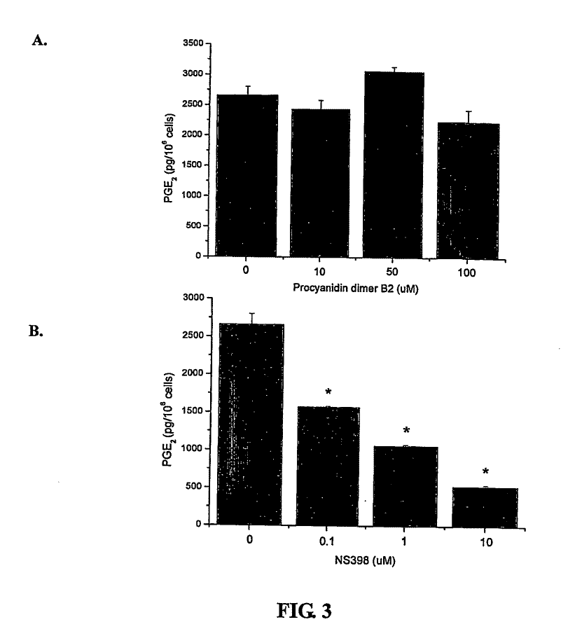

To determine if B2 had a direct effect on COX-2 enzyme activity, a baculovirus-expressed human recombinant COX-2 in a cell-free assay was used. FIG. 3 showed that B2 had no inhibitory effect on PGE2 synthesis, a product of COX-2 enzyme activity, in contrast to the NS398 positive control.

### Example 2

### Effect of Procyanidin Dimers (A1, B1 and B2) and Flavanols ((−)-Catechin and (+)-Epicatechin) on COX-2 Expression

### Materials

Phorbol 12-myristate 13-acetate (PMA), lipopolysaccharide (LPS), and N-formyl-methionyl-leucyl-phenylalanine (fMLP), were obtained from Sigma (St. Louis, Mo.). Chemicals employed for gel electrophoresis were purchased from Bio-Rad (Hercules, Calif.). Trypsin sequencing grade was obtained from Promega (Southampton, United Kingdom). EDTA, EGTA, and PMSF were purchased from Amresco (Solon, Ohio). Flavanols (+)-catechin and (−)-epicatechin were purchased from Sigma, and (−) catechin and (+)-epicatechin were prepared by thermally-treating (+)-catechin and (−)-epicatechin, respectively in an aqueous solution as described above. Procyanidin dimer B2 was prepared from cocoa by solvent extraction, using gel permeation chromatography, followed by further purification/isolation of a dimer enriched fraction using Normal-Phase HPLC (described in detail in Adamson et al., J. Ag. Food Chem., 1999, 47 (10):4184-4188), see also U.S. Pat. No. 5,554,645, both of which are hereby incorporated herein by reference. This material was then passed over a C18 column to further enrich B2 dimer (98.3%) in the fraction which was used in the experiments described below. Procyanidin dimer B1 was prepared synthetically as described in U.S. Pat. No. 6,420,572, which is hereby incorporated herein by reference. A1 dimer was prepared from peanut skins as described in U.S. application Ser. No. 11/045,648 published as US 2005/0164956, which is hereby incorporated herein by reference.

### Cell Culture, RNA Extraction and Polymerase Chain Reaction (RT-PCR)

The human monocyte line U937 was obtained from the cell bank in Shanghai Institute of Biological Sciences, Chinese Academy of Sciences. Monocytic cells of 3-4 passages were grown to confluence in RPMI 1640 (GIBCO-BRL, Glasgow, Scotland) containing 10% fetal bovine serum at 37° C. in a 5% CO₂ humidified incubator during all experimental procedures. Macrophages were pretreated with procyanidins B1 dimer, B2 dimer, A1 dimer, and flavanols (−)-catechin and (+)-epicatechin (each 10) for 2 h before LPS (1 μg·ml⁻¹) priming or fMLP (10 μM) stimulation for 2 h. The normal macrophages without LPS or fMLP treatment were considered as controls. They were then differentiated to macrophages in the medium containing 100 nM PMA and allowed to adhere for 48 h, after which they were fed with PMA-free medium and cultured for 24 h prior to use. Cells from different groups were collected and total RNA was prepared with TRI-REAGENT-LS extraction kit The expression of RNAs was determined by RT-PCR. Complementary DNA was created from RNA using TrueScript MMLV reverse transcriptase and oligo d (T) 18 primers. 5 μg RNA was included in each reaction. The primers of COX-2 and GAPDH are shown in the following table.

| | | Products |
|---|---|---|
| Gene Name | Primer sequnce | (bp) |
| COX-2 | sense: | 503 |
| | 5′-TATACTAGAGCCCTTCCTCCTGTGCC-3′ | |
| | [SEQ ID NO: 5] | |
| | antisense: | |
| | 5′-ACATCGCATACTCTGTTGTGTTCCC-3′ | |
| | [SEQ ID NO: 6] | |
| GAPDH | sense: | 200 |
| | 5′-AAGAAGGTGGTGAAGCAGGC-3′ | |
| | [SEQ ID NO: 7] | |
| | antisense: | |
| | 5′-CCACCACCCTGTTGCTGTAG-3′ | |
| | [SEQ ID NO. 8] | |

### Statistical Analysis of Data

The data are presented as means±SD and compared with ANOVA and least significant difference test using SPSS statistical program. The level of the statistical significance was set at P<0.05.

### Results

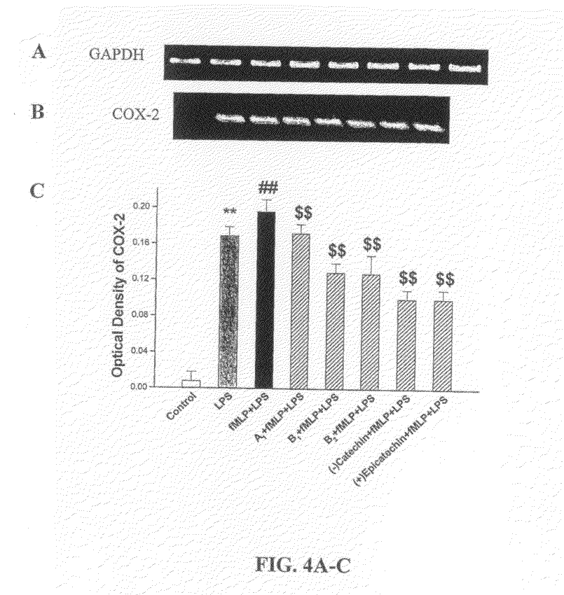

The anti-inflammatory effects of B1 dimer, B2 dimer, A1 dimer, (−)catechin and (+)-epicatechin were investigated. As shown in FIGS. 4 A-C, (−)-catechin, (+)-epicatechin, A1, B1 and B2 showed significant inhibition of the mRNA expression of COX-2. Further, the order of suppression potency of the COX-2 mRNA expression induced by LPS and fMLP was: (−)-catechin, (+)-epicatechin >B1, B2>A1 (FIG. 4C).

What is claimed is:

1. A method of treating inflammation and/or inflammation-related or associated disease or condition, which comprises administering, to a subject in need thereof, a composition comprising an effective amount of a compound having the formula Aₙ, or a pharmaceutically acceptable salt or derivative thereof (including oxidation products, methylated derivatives and glucuronidated derivatives ): 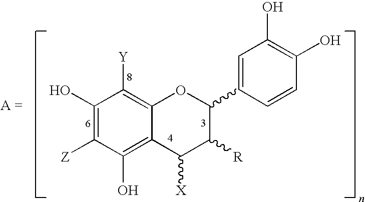
wherein
n is an integer from 2 to 18;
R and X each have either α or β stereochemistry;
R is OH, O-sugar or O-gallate;
the substituents of C-4, C-6 and C-8 are X, Z and Y, respectively, and bonding of monomeric units occurs at C-4, C-6 or C-8;
when any C-4, C-6 or C-8 are not bonded to another monomeric unit, X, Y and Z independently are hydrogen or a sugar; and
the sugar is optionally substituted with a phenolic moiety at any position, for instance, via an ester bond; and
wherein the subject is a human or a veterinary animal and is sensitive to a selective COX-2 inhibitor and/or COX-nonselective NSAID.

2. The method of claim 1, wherein the subject is a human.

3. The method of claim 2, wherein the composition is a pharmaceutical composition.

4. The method of claim 3, wherein n=2-12.

5. The method of claim 3, wherein n=2-5.

6. The method of claim 3, wherein n=2.

7. The method of claim 1, wherein R is OH.

8. The method of claim 7, wherein the subject is a human.

9. The method of claim 8, wherein n=2-12.

10. The method of claim 8, wherein n=2-5.

11. The method of claim 8, wherein n=2.

12. The method of claim 11, wherein the compound is dimer B2.

13. The method of claim 3, wherein the subject suffers from arthritis.

14. The method of claim 3, wherein the subjects suffers from a gastrointestinal disease or condition; a respiratory/pulmonary disease or condition; a musculoskeletal disease or condition; a dermal disease or condition; an allergic disease or condition; pain associated with dysmenorrhea, menstrual cramps, headache, toothache, low back and neck pain; an ocular disease or condition; a diabetes-associated condition; fever; systemic lupus erythematosis; an inflammation-associated with burns; and/or an inflammatory disease or condition affecting multiple organs .

15. The method of claim 3, wherein the subject suffers from osteoporosis.

16. The method of claim 3, wherein the subject suffers from diabetes-associated inflammatory conditions.

17. The method of claim 13, wherein the compound is dimer B1 or B2.

18. The method of claim 14, wherein the compound is dimer B1 or B2.

19. The method of claim 15, wherein the compound is dimer B1 or B2.

20. The method of claim 16, wherein the compound is dimer B1 or B2.

21. A method of treating inflammation and/or inflammation-related or associated disease or condition, which comprises administering, to a subject in need thereof, a composition comprising an effective amount of a compound having the formula: 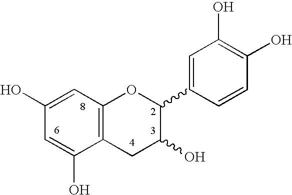
or a pharmaceutically acceptable salt thereof
wherein the subject is a human or a veterinary animal and is sensitive to a selective COX-2 inhibitor and/or COX-nonselective NSAID.

22. The method of claim 21, wherein the subject is a human.

23. The method of claim 21, wherein the compound is (+)-epicatechin.

24. The method of claim 21, wherein the compound is (−)-catechin.


SEQUENCE LISTING (8 sequences)

SEQ ID NO: 1 - DNA, 20 bp, Artificial Sequence
  Chemically synthesized
gggcaaagactgcgaagaag

SEQ ID NO: 2 - DNA, 20 bp, Artificial Sequence
  Chemically synthesized
cccatgtgacgaaatgactg

SEQ ID NO: 3 - DNA, 20 bp, Artificial Sequence
  Chemically synthesized
acggatttggtcgtattggg

SEQ ID NO: 4 - DNA, 20 bp, Artificial Sequence
  Chemically synthesized
cgctcctggaagatggtgat

SEQ ID NO: 5 - DNA, 26 bp, Artificial Sequence
  Chemically synthesized
tatactagagcccttcctcctgtgcc

SEQ ID NO: 6 - DNA, 25 bp, Artificial Sequence
  Chemically synthesized
acatcgcatactctgttgtgttccc

SEQ ID NO: 7 - DNA, 20 bp, Artificial Sequence
  Chemically synthesized
aagaaggtggtgaagcaggc

SEQ ID NO: 8 - DNA, 20 bp, Artificial Sequence
  Chemically synthesized
ccaccaccctgttgctgtag